(19) United States
(12) Patent Application Publication
(10) Pub. No.: US 2006/0052754 A1
(43) Pub. Date: Mar. 9, 2006

(54) Thumb trigger syringe pole

(76) Inventor: Douglas Wade Fields, Columbus, OH (US)

Correspondence Address: DOUGLAS W. FIELDS, 3655 BEECHTON RD., COLUMBUS, OH, 43232, US

(21) Appl. No.: 10/933,943

(22) Filed: Sep. 4, 2004

(51) Int. Cl. A61M 5/315

(52) U.S. Cl. 604/218

(57) ABSTRACT

The invention is a syringe pole device used to inject dangerous animals while operating the plunger of the syringe in and out from the rear of the pole. The invention consists of a specific size outer tube with a threaded fitting permanently attached on the front end. A specific diameter and length of inner tube with a specific size and shape knob permanently attached to the back of the tube serves as a push/pull knob. After inserting the inner tube through the outer tube, a specific size flat washer is affixed to the front of the inner tube by using a specific size sheet metal screw. A specific size plunger is stretched over the washer and a specific size syringe is slid over the plunger and twisted onto the threaded fitting that is attached to the outer tube, thus completing the syringe pole device.

### BACKGROUND OF THE INVENTION

1. Field of the Invention

This invention relates to the ability to inject an anesthesia or sedation fluid into caged or restrained animals from a relative safe distance. This invention is designed for nuisance wildlife control and veterinary applications.

It is common for nuisance wildlife trappers and veterinarians to sedate dangerous animals by means of injection. The invention allows the user to efficiently perform injection procedures on dangerous animals with a minimum amount of time and effort.

2. Description of the Prior Art

Injecting and immobilizing dangerous animals can be a hazardous and time consuming job. Some common methods of injecting dangerous animals requires the user to fill a syringe with the desired fluid to be used and then attach the filled syringe onto the end of a pole or into a compartment made to accept the syringe on the end of a pole and secure it in place. The syringe is then pushed up against the target animal to insert the needle of the syringe into the animal and then the user must push harder against the animal to depress the plunger of the syringe to inject the animal. The disadvantages of these methods is the lack of control during the injection process and the large amount of stress that is placed on the animal. Another disadvantage is the need to remove the empty syringe from the pole to refill it and then remount it onto the pole for every injection made.

### SUMMARY

The present invention provides the user with the ability to efficiently perform injection procedures on dangerous animals from a relative safe distance. The target animal is caged or otherwise restrained for the injection process. First, install the plunger onto the washer on the front end of the inner tube. Slide an empty syringe over the plunger and twist the syringe onto the threaded fitting on the outer tube on front of the syringe pole. Then, place the needle onto the syringe and fill the syringe by placing the needle into the container of fluid to be used. Draw the fluid into the syringe by pulling out on the inner tube on the rear of the pole using the push/pull knob. Gently insert the needle into the target animal and while holding the pole stationary, push in on the inner tube on the rear of the pole to depress the plunger, thus injecting the animal.

One advantage of the present invention is that the plunger of the syringe is operated in and out from the rear of the pole making it safer and more efficient for the user, and less stressful on the animal.

Another advantage of the present invention is that a syringe and plunger can be attached to the syringe pole by hand without the need for tools.

Yet another advantage of the present invention is that the syringe can be filled and emptied with ease numerous times when multiple shots are required, while the syringe stays mounted on the syringe pole.

Still another advantage is that it comes in different lengths and sizes to accommodate the various uses for the nuisance wildlife control and veterinary applications.

The thumb trigger syringe pole device allows the user to fill a syringe and gently inject dangerous animals while working the plunger of the syringe in and out from the rear of the syringe pole. All the parts can be assembled by hand with no tools needed.

### DRAWING FIGURES

FIG. 1 shows a perspective view of a completely assembled syringe pole device. A portion of the outer tube is cut away to show clarity.

FIG. 2 shows a perspective view of each part of the syringe pole device.

FIG. 3 shows an exploded view of the plunger, syringe and needle assembled on the syringe pole device.

FIG. 4 shows an exploded view of the plunger being attached to the front end of the syringe pole device.

### REFERENCE NUMERALS IN DRAWING

10 Inner flanged tube
12 Outer flanged tube
14 Threaded fitting
16 Flat washer
18 Sheet metal screw
20 Plunger
22 Standard veterinary syringe
24 Disposable needle
26 Push/Pull knob

### DETAILED DESCRIPTION OF THE PREFERRED EMBODIMENT

The following represents a detailed description of the preferred embodiment of the invention, presently considered to be the best made invention when applied to nuisance wildlife control and veterinary applications.

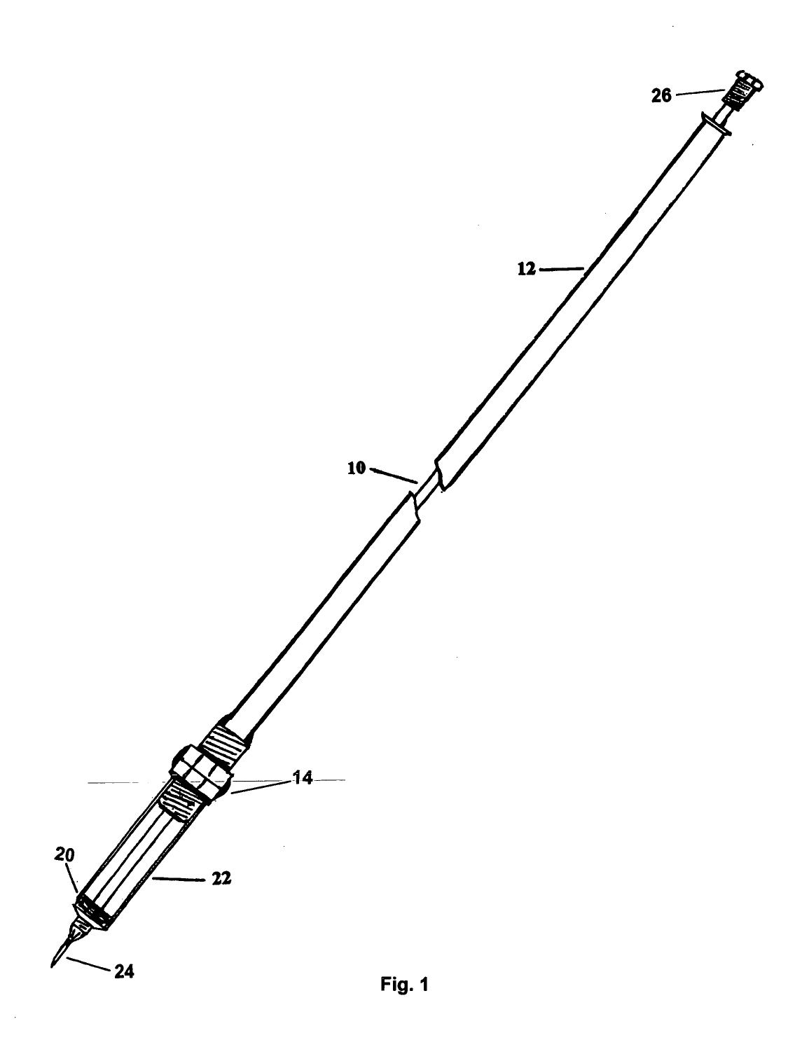

FIG. 1 shows a perspective view of the thumb trigger syringe pole device in accordance with the preferred embodiment of the invention seen as it would appear to an individual using the syringe pole.

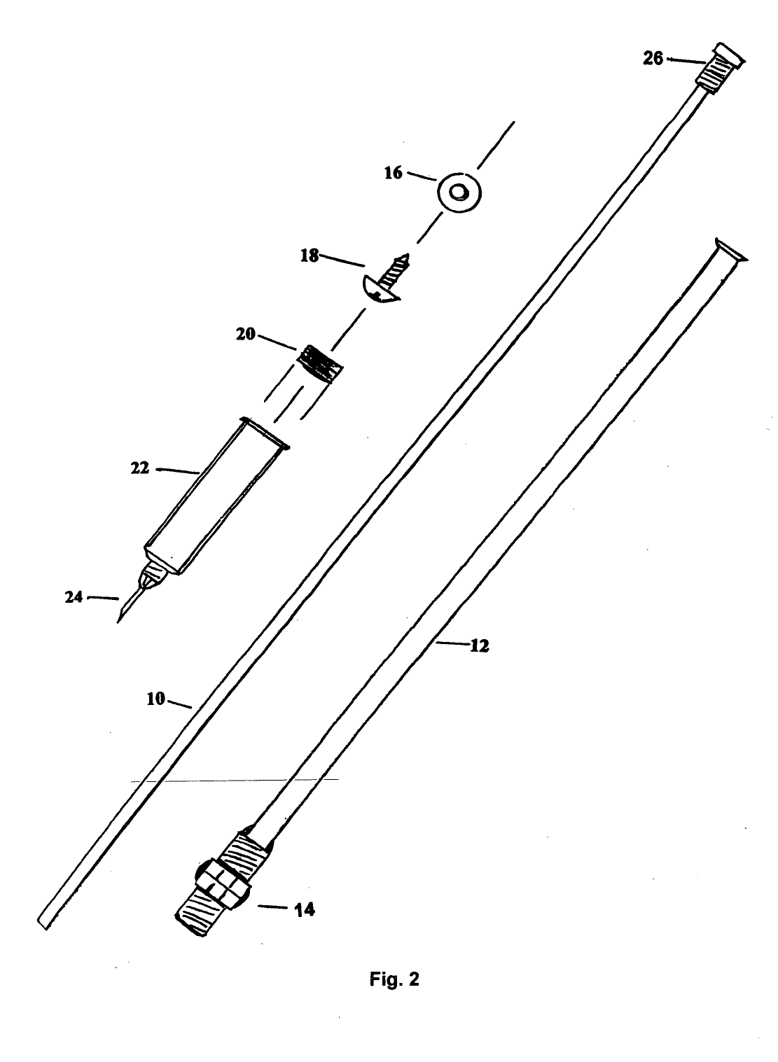

FIG. 2 shows a view of the each component part of the syringe pole device.

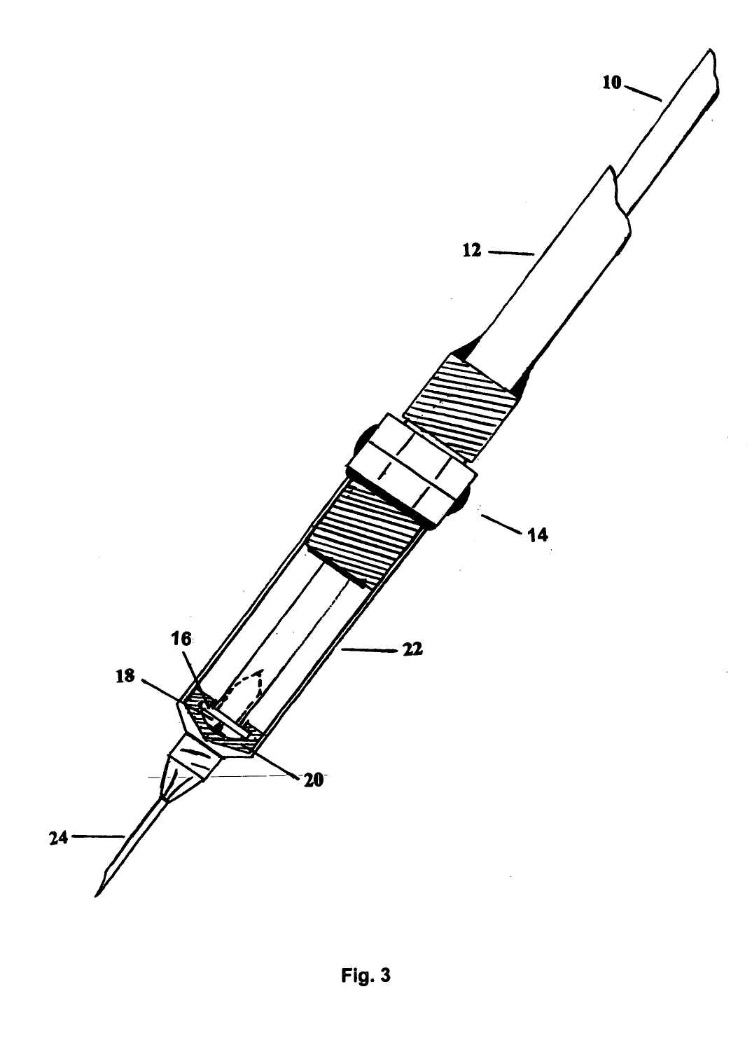

FIG. 3 shows an enlarged view of the plunger, syringe and needle mounted on the front end of the syringe pole device.

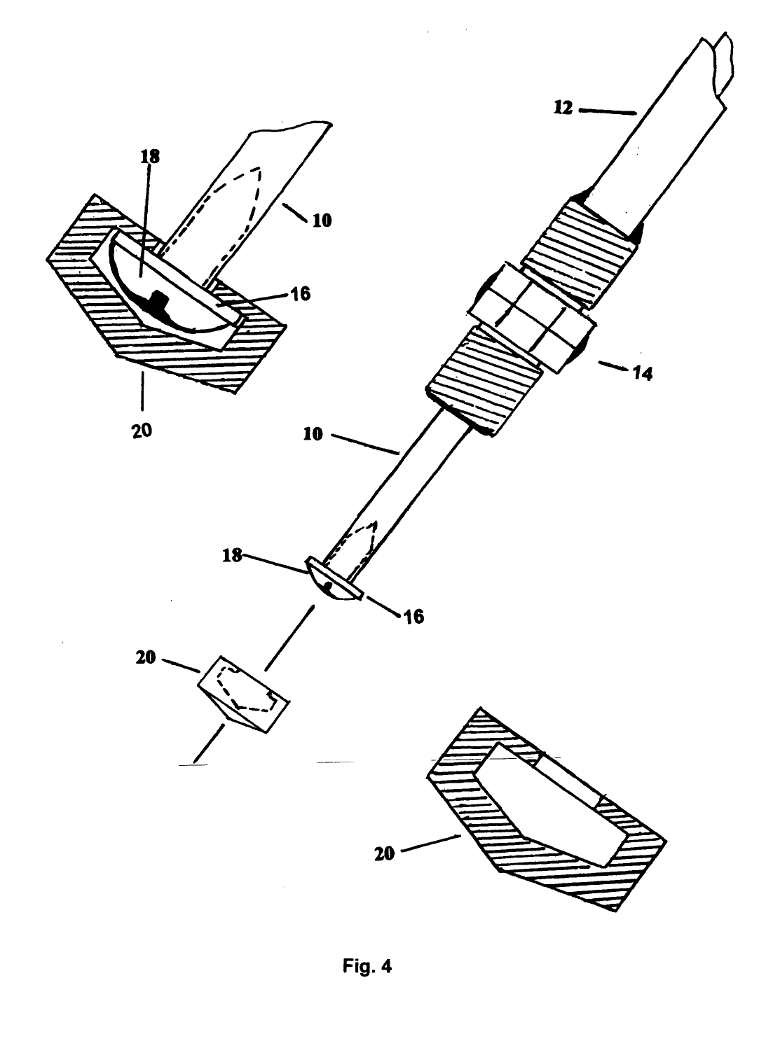

FIG. 4 shows a view of the front end of the syringe pole with the plunger being inserted over the flat washer and sheet metal screw.

Advantages

A number of advantages of the thumb trigger syringe pole are:
(a) The small amount of time and effort required to securely attach a plunger and syringe onto the front end of the pole.
(b) The elimination of the various clamps and straps commonly used to hold the syringe inside of or onto the end of a pole.
(c) The pole allows the user to attach a syringe to the front end of the pole by hand and operate the plunger of the syringe in and out from the rear of the pole.
(d) The ability to fill, empty and refill the syringe numerous times while the syringe remains attached to the pole, thus removing the need to disassemble the syringe from the pole to refill the syringe.
(e) It can be manufactured for different size syringes and in different lengths to accommodate the various uses for nuisance wildlife control and veterinary applications.
   Operation

The first thing that must be done when using the syringe pole device is to place the plunger 20 onto the flat washer 16 on front of the inner tube 10.

Slide an empty syringe 22 over the plunger 20 and twist onto the threaded fitting 14 that is affixed to the outer tube 12 on the front end of the syringe pole.

Place the needle 24 on the syringe 22 and insert the needle 24 into the injection fluid to be used.

Then, while grasping the outer tube 12, slowly pull out on inner flanged tube 10 by using the push/pull knob 26, thus pulling out the plunger and filling the syringe 22.

The syringe pole device is then maneuvered into position to inject the target animal while grasping the outer flanged tube 12 and gently inserting the needle 24 into target animal and slowly depress the push/pull knob 26 on the rear of the inner flange tube 10 thus pushing the plunger 20 inside the syringe 22 expelling the contents of the syringe 22 and injecting the target animal.

### CONCLUSIONS, RAMIFICATIONS AND SCOPE

Accordingly, the reader will see that this invention enables the user to easily and efficiently perform injection procedures on dangerous animals while operating the plunger of the syringe in and out from the rear of the pole. The syringe pole device provides a highly reliable, yet economical means of performing injections with a minimum amount of time and effort. Furthermore, the thumb trigger syringe pole has the additional advantages in that:
The syringe can be filled, emptied and refilled numerous times while the syringe remains attached to the pole, thus removing the need to disassemble the syringe from the pole to refill the syringe;
It provides a safer more efficient method of performing injection procedures on dangerous animals;
It can be made for different size syringes and in different lengths to accommodate different uses;
All parts can be assembled onto the syringe pole device by hand without the need for tools;
It is made from light weight rust resistant materials.
   Alterations or modifications may occur to others upon a reading and understanding of this specification. It is intended to include all such modifications and alterations insofar as they come within the scope of the appended claims and their legal equivalents, rather than by the examples given.

I claim:

1. A syringe pole device used to inject animals while operating the plunger of the syringe in and out from the rear of the syringe pole, said syringe pole device comprises:
a) an outer tube of a specific diameter and length that is determined by the size of said syringe pole device that it is made for, said outer tube having a specific size flange formed on both ends;
b) a threaded fitting of a measured inside diameter, outside diameter and length that is determined by the size of said syringe pole device that it is made for;
c) that said threaded fitting is positioned against said flange on front of said outer tube and is permanently affixed by means of welding and serves as a mount for said syringe;
d) an inner tube of a specific diameter as such that it will pass freely through the inside of said outer tube and of a length that is determined by the size of said syringe pole device that it is made for, said inner tube having a specific size and shaped knob affixed to the back and serves as a push/pull knob;
e) a flat washer of a measured outside diameter with a specific size hole and thickness is affixed to the front end of said inner tube by means of a specific size sheet metal screw which serves as a mount for said plunger.

2. A combination syringe and syringe pole device;
said syringe pole device allows the user to attach said syringe onto said outer tube and operate said plunger of said syringe from the rear of said syringe pole device;
said syringe pole device comprises:
a) an outer tube of a specific diameter and length that is determined by the size of said syringe pole device that it is made for, said outer tube having a specific size flange formed on both ends;
b) a threaded fitting of a measured inside diameter, outside diameter and length that is determined by the size of said syringe pole device that it is made for;
c) that said threaded fitting is positioned against said flange on front of said outer tube and is permanently affixed by means of welding and serves as a mount for said syringe;
d) an inner tube of a specific diameter as such that it will pass freely through the inside of said outer tube and of a length that is determined by the size of said syringe pole device that it is made for, said inner tube having a specific size and shaped knob affixed to the back and serves as a push/pull knob;
e) a flat washer of a measured outside diameter with a specific size hole and thickness is affixed to the front end of said inner tube by means of a specific size sheet metal screw which serves as a mount for said plunger.

3. Said combination of claim 2 further comprises said plunger of part e) is attached to said flat washer on the front of said inner tube and is moved in and out inside of said syringe by moving the push/pull knob on the rear of said inner tube.
